(19) United States
(12) Patent Application Publication
(10) Pub. No.: US 2005/0089944 A1
(43) Pub. Date: Apr. 28, 2005

(54) High sensitivity amperometric biosensor with side-to-side hybrid configuration

(75) Inventors: Paul Shieh, Fremont, CA (US); Ching-Hsin Cho, San Chung (TW)

Correspondence Address: C.P. Chang;c/o Pacific Law Group LLP, Suite 525, 224 Airport Parkway, San Jose, CA, 95110, US

(73) Assignee: BioMedix, Inc., Fremont, CA (US)

(21) Appl. No.: 10/695,273

(22) Filed: Oct. 27, 2003

(51) Int. Cl. C12Q 1/54

(52) U.S. Cl. 435/14

(57) ABSTRACT

An amperometric glucose biosensor comprises a sensing electrode and a reference electrode arranged in a side-by-side parallel configuration on an electrically insulating sheet. A passive cover electrode is placed over the side-by-side sensing electrode and reference electrode so that the active surface of the passive cover electrode opposes the active surfaces of the side-by-side electrodes. Physical contact between the passive covering electrode and the side-by-side electrodes is prevented by insulating spacers. The sensing electrode comprises a conductive graphite track coated with a formulation comprising a redox mediator and enzyme and the reference electrode is a parallel track comprising an Ag/AgCl formulation while the passive cover electrode comprising a conductive graphite track coated with a formulation comprising the same redox mediator as used in the sensing electrode but not including an enzyme. An opening either located in the middle or at one side of the passive cover electrode allows a liquid test sample to be introduced into the sensor. The biosensor of the present invention exhibits a sensitivity and response time equal to or surpassing that of the simple face-to-face configuration while can be efficiently manufactured and permit use with conventional electrical connectors.

### FIELD OF THE INVENTION

This invention deals with improvements in amperometric biosensors, particularly biosensors designed to detect glucose in biological fluids and other media, in order to provide a high sensitivity device that can be efficiently manufactured and conveniently used.

### BACKGROUND OF THE INVENTION

Guo, et al, in U.S. Pat. No. 6,033,866, which is herein incorporated by reference, disclose a biosensor for the rapid determination of glucose in bodily fluids such as blood and urine that employs a bimediator, bienzyme system in a biosensor having a face-to-face sandwich configuration. In this biosensor one mediator is incorporated into the conductive layer of the working electrode and the second mediator is contained in a reagent strip that is sandwiched between the active surface of the working electrode and the active surface of the reference electrode. Although highly effective for the rapid detection of glucose in small samples the use of two mediators in a biosensor having a simple sandwich configuration is inefficient to manufacture. As such biosensors are generally in practice plugged into a readout device, cost considerations necessitate that the configuration of the electrical connections to the electrodes be such that sensor can be used with electrical connectors having standard configurations known and commonly used in the art. Such connectors are commonly designed to receive electrical connections printed with conductive substances such as copper or conductive inks on the surface of a circuit board. Although the simple face-to-face configuration of the glucose sensor disclosed in U.S. Pat. No. 6,033,866 provides excellent sensitivity, to use it with commonly available electrical connectors, one of the electrodes, generally the reference electrode, must be electrically connected to a conductive strip that is in the same plane and parallel to the second electrode, generally the working or sensing electrode. Such electrical connections which can be made with conductive tape and the like, prove to be cumbersome in manufacturing and can produce unreliable results thereby greatly increasing the manufacturing cost of such sensors. On the other hand, biosensors employing a simple side-by-side configuration, in which both the working electrode and the reference electrode are printed as parallel strips on an insulating surface, while adapted to easy and convenient use with conventional electrical connectors, do not provide the high sensitivity short response time of the face-to-face sandwich configuration. There is therefore a need for biosensor, particularly a glucose sensor that provides the high sensitivity and short response time of the face-to-face sandwich configuration and can be economically and efficiently manufactured for use with standard electrical connectors known in the art.

### BRIEF DESCRIPTION OF THE INVENTION

The present invention is directed to meeting the foregoing needs by providing an amperometric glucose biosensor based on a one mediator, one enzyme redox system having a configuration that provides the high sensitivity and fast response time of the face-to-face configuration while permitting easy and convenient use with conventional electrical connectors. In the biosensor of the present invention a sensing electrode and a reference electrode are arranged in a side-by-side parallel configuration on an electrically insulating sheet. In one embodiment the sensing electrode comprises a conductive graphite track coated with a formulation comprising a redox mediator and enzyme and the reference electrode is a parallel track comprising an Ag/AgCl formulation. A further component of the biosensor is a passive cover electrode comprising a conductive graphite track on an insulating sheet, with the graphite track coated with a formulation comprising the same redox mediator as used in the sensing electrode but not including an enzyme. Various redox compounds, ions and complexes such as CN, Fe(CN)₆⁻³, I, Co(NH₃)₆⁺³, Sn⁺³,S2- or Tl⁺³ can be used as the redox mediator. The passive cover electrode is placed over the side-by-side sensing electrode and reference electrode so that the active surface of the passive electrode opposes the active surfaces of the side-by-side electrodes. Physical contact between the passive covering electrode and the side-by-side electrodes is prevented by insulating spacers. An opening either located in the middle or at one side of the passive cover electrode allows a liquid test sample to be introduced into the sensor. The side-by-side electrodes are easily connected to standard electrical connectors via conductive connecting tracks coasted on the insulating sheet, while the passive cover electrode has no separate electrical connector but becomes part of the active sensing cell when a liquid sample is introduced into the sensor. The biosensor of the present invention exhibits a sensitivity and response time equal to or surpassing that of the simple face-to-face configuration disclosed in U.S. Pat. No. 6,033,866, while the side-by-side active electrode elements can be efficiently manufactured and permit use with conventional electrical connectors without the need of additional unreliable manufacturing steps.

### BRIEF DESCRIPTION OF THE DRAWINGS

The current invention will be better understood and the nature of the objectives set forth above will become apparent when consideration is given to the following detailed description of the preferred embodiments. For clarity of explanation, the detailed description further makes reference to the attached drawings herein:

FIG. 1A is a top view of an embodiment of the biosensor of the present invention which is a hybrid of side-by-side and face-to-face electrode configurations;

FIG. 1B is a cross-sectional view along line I-I of the biosensor of FIG. 1A;

FIG. 1C is an exploded view of along line II-II of the biosensor of FIG. 1A;

FIG. 1D depicts an array of side-by-side electrode components and an array of passive cover electrode screen printed on an insulating base as may be used in the manufacture of biosensor of FIG. 1A;

FIG. 2A is a top view of another embodiment of the biosensor of the present invention in which an opening is provided on at one side of the biosensor;

FIG. 2B is an exploded view along line I-I of the biosensor of FIG. 2A;

FIG. 3 is a graphical comparison of the performance of a hybrid glucose sensor versus a conventional side-by-side glucose sensor;

FIG. 4 is a plot of current response versus glucose concentration for a hybrid glucose sensor;

FIG. 5 is a plot of current response versus glucose concentration in whole blood for a hybrid glucose sensor having mediator in the passive electrode and a hybrid glucose sensor not having mediator in the passive electrode; and

FIG. 6 is a concentration vs concentration scatter diagram of the results obtained on whole blood sample using a glucose biosensor embodiment of the present invention and a commercially accepted blood glucose test.

### DESCRIPTION OF THE PREFERRED EMBODMIMENTS

The abbreviations used in the description of different embodiments of the invention have been discussed by Guo, et al, in U.S. Pat. No. 6,033,866, which is herein incorporated by reference.

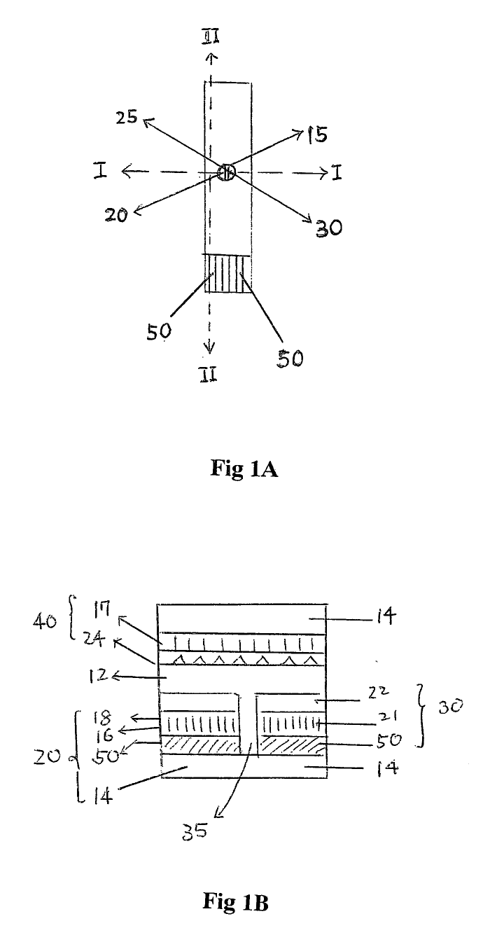

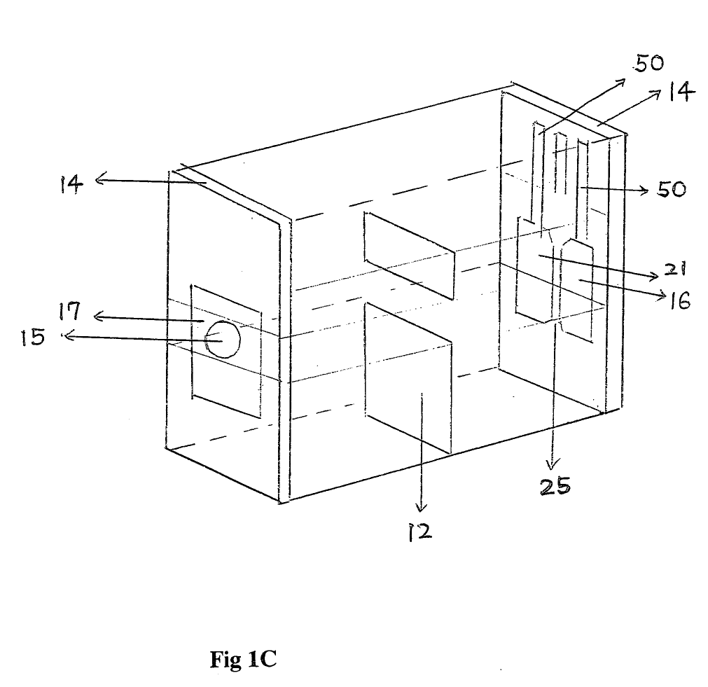

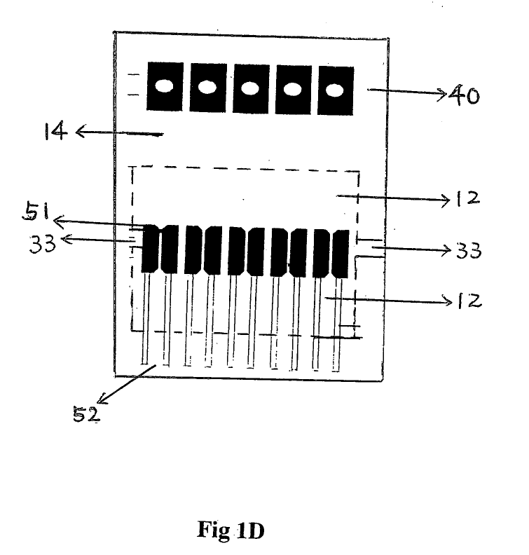

FIG. 1A is a top view of an embodiment of the amperometric hybrid biosensor 10 of the present invention. The hybrid biosensor 10 generally comprises a sensing electrode 20, a reference electrode 30 and a passive cover electrode 40. As depicted in FIG. 1A portion of sensing electrode 20 and reference electrode 30 are visible through opening 15 in passive cover electrode 40. Also visible through opening 15 of FIG. 1A is a separation 25 that separate sensing electrode 20 and reference electrode 30. Separate electrically conductive tracks 50 are electrically connected to sensing electrode 20 and reference electrode 30.

FIG. 1B is a cross-sectional view through I-I of the sensing electrode and passive electrode of biosensor 10 of FIG. 1A. Sensing electrode 20 comprises an insulating base 14 that supports electrically conductive track 50 which is connected to conductive electrode base 16 also supported by insulating base 14. Conductive electrode base 16 of sensing electrode 20 is coated with sensing electrode formulation 18. Non-electrically conductive spacers 12 are disposed at the ends of coating 18 so that a cavity 35 is formed when passive cover electrode 40 is placed over sensing electrode 20 and reference electrode 30. Passive cover electrode 40 rests on spacers 12 so that sensing electrode 20 and passive cover electrode 40 do not come into physical contact. Passive cover electrode formulation 24 coats conductive electrode base 17 of passive cover electrode 40. Conductive electrode base 17 is coated on insulating base 14 of passive electrode 40.

FIG. 1C is an exploded view along II-II of the biosensor 10 of FIG. 1A. Reference electrode 30 comprises an insulating base 14 that supports electrically conductive track 50 which is connected to conductive electrode base 21 that is also supported by insulating base 14. Conductive electrode base 21 of reference electrode 30 is coated with reference electrode formulation 22. Non-electrically conductive spacers 12 are disposed at the ends of coating 22 so that a cavity 35 is formed when passive cover electrode 40 is placed over sensing electrode 20 and reference electrode 30. Passive cover electrode 40 rests on spacers 12 so that reference electrode 30 and passive electrode 40 do not come into physical contact. Passive cover electrode 40 does not have a separate conductive electrical connection as sensing electrode 20 and reference electrode 30 have. Passive cover electrode 40 is activated when a liquid sample is introduced into biosensor 10.

FIG. 1D depicts an array of side-by-side electrode components 51 and an array of passive cover electrode screen printed on an insulating base 14 as may be used in the manufacture of biosensor 10. As depicted in FIG. 1D, insulating spacers 12 are arranged in parallel fashion across adjacent side-by-side electrode components 51 to define a reaction zone 33, which when covered with passive cover electrode 40 forms cavity 35 as depicted in FIGS. 1B and 1C. Opening 15 in passive cover electrode 40 through which sample is introduced is not depicted in FIG. 1D.

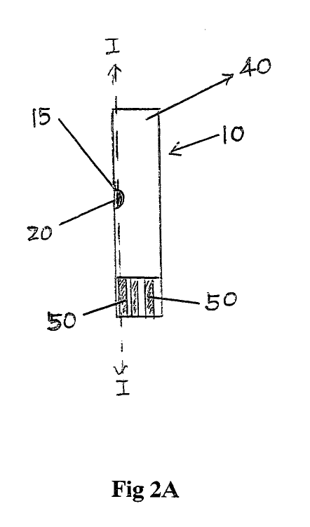

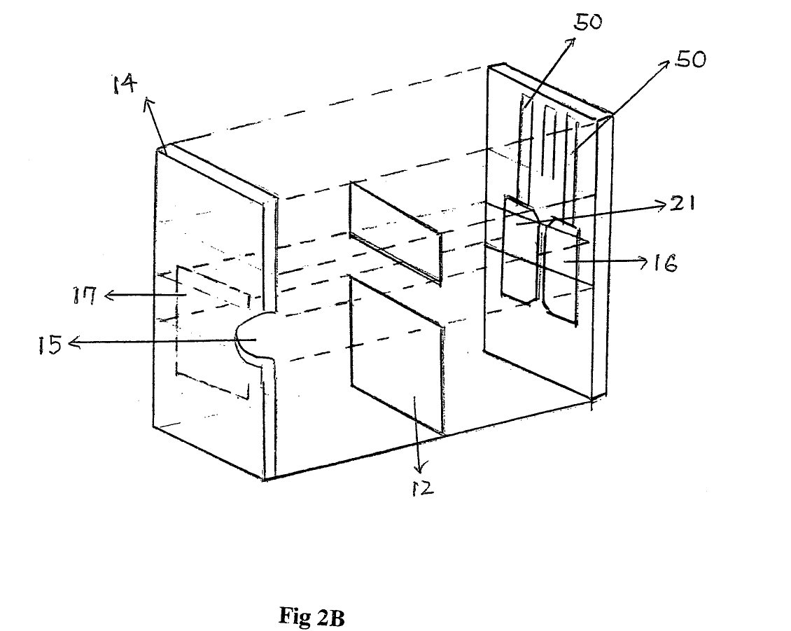

Referring jointly to FIG. 2A and FIG. 2B, there is shown another embodiment of the amperometric hybrid biosensor according to the present invention. FIG. 2A is a top view of the amperometric hybrid biosensor 10 and FIG. 2B is an exploded view along line I-I of the biosensor of FIG. 2A. The biosensor 10 comprises a sensing electrode 20, a reference electrode 30 (depicted in FIG. 2B) and a passive cover electrode 40. As depicted in FIG. 2A, only a portion of sensing electrode 20 is visible through opening 15 in the passive cover electrode 40. While not visible through opening 15 of FIG. 2A, a separation 25 is provided to separate sensing electrode 20 and reference electrode 30. Separate electrically conductive tracks 50 are electrically connected to sensing electrode 20 and reference electrode 30.

Like the biosensor described above and depicted in FIGS. 1A-1D, the sensing electrode 20 according to the another embodiment comprises an insulating base 14 that supports electrically conductive track 50 which is connected to conductive electrode base 16 that is also supported by insulating base 14. Conductive electrode base 16 of sensing electrode 20 is coated with sensing electrode formulation 18. Non-electrically conductive spacers 12 are disposed at the ends of coating 18 so that a cavity 35 is formed when passive cover electrode 40 is placed over sensing electrode 20 and reference electrode 30. Passive cover electrode 40 rests on spacers 12 so that sensing electrode 20 and passive cover electrode 40 do not come into physical contact. Passive cover electrode formulation 24 coats conductive electrode base 17 of passive cover electrode 40. Conductive electrode base 17 is coated on insulating base 14 of passive electrode 40.

Likewise, the reference electrode of the another embodiment 30 comprises an insulating base 14 that supports electrically conductive track 50 which is connected to conductive electrode base 21 that is also supported by insulating base 14. Conductive electrode base 21 of reference electrode 30 is coated with reference electrode formulation 22. Non-electrically conductive spacers 12 are disposed at the ends of coating 22 so that a cavity 35 is formed when passive cover electrode 40 is placed over sensing electrode 20 and reference electrode 30. Passive cover electrode 40 rests on spacers 12 so that reference electrode 30 and passive electrode 40 do not come into physical contact. Passive cover electrode 40 does not have a separate conductive electrical connection as sensing electrode 20 and reference electrode 30 have. Passive cover electrode 40 is activated when a liquid sample is introduced into biosensor 10.

Accordingly, the embodiment as depicted in FIGS. 2A-2B provides an amperometric hybrid biosensor comprising all the elements and characteristic features identical to the biosensor described in FIGs 1A-1D except that the opening 15, which is to receive aqueous blood sample, is located on one side, rather than on the middle, of the passive cover electrode 40. Through this arrangement, only a portion of the conductive electrode base 16 of the sensing electrode 20 is visible from and thus exposed by the opening 15. While the opening 15 could be in varying sizes and shapes, it is preferable that the opening 15 as depicted in FIGS. 2A-2B is no more than half of the size of the opening as depicted in FIGS. 1A-1D. The main purpose of having an opening placed on one side of the passive cover electrode 40 exposing only a portion of the sensing electrode 20 is to take advantage of the capillary phenomenon provided by the biosensor that enables the blood to penetrate the whole reaction area within a short period of time. In addition, since the aqueous blood sample is introduced directly onto the sensing electrode 20, only a tiny amount of blood drop in the range of about 0.5 to 1 μl (IS THIS CORRECT?) shall be sufficient to meet the necessary minimum amount for the reaction. In a sense, the patient has much less pain when sampling the blood from finger tip employing the amperometric hybrid biosensor according to the present invention.

The insulating base 14 (or non-conductive support member 14) may typically be any cohesive non-conductor such as any non-conductive film or sheet forming polymeric material, ceramics, glass, paper, cardboard. The preferred thickness of the non-conductive support material is from about 5 mil to about 10 mil. Polymeric material, particularly non-conductive polymerics in the form of films or thin sheets are preferred as thy may be readily cut to strips of suitable size. In practice insulating base 14 is generally a polymeric film or sheet. Any non-conductive polymeric film or sheet such as polyvinyl chloride, polyester, polycarbonate, vinyl acetate copolymer, nylon, poly (1,4-butleneterephthalate), cellulose propionate, ethylene/acrylic acid copolymer, polybutadiene, polyethylene, polypropylene, polyimide, acrylic film, polyurethane, polystyrene, and polyvinyl fluoride may be used. Polyester film such as Mylar® is preferred as it is readily available and easily handled.

Conductive tracks 50 may be comprised of any electrically conductive material known in the art such as metallic silver, silver salts or mixtures thereof, conductive graphite or carbon, copper, platinum, cobalt, nickel, gold and electrically conductive polymers. Conductive tracks 50 may also differ in composition within the same biosensor. For example, one electrode may 20 use a conductive track 50 comprised of silver while the other may use a conductive track 50 comprised of conductive graphite. Formulations of silver and silver chloride are preferred as they are commercially available in the form of inks and provide excellent conductivity as thin films that may be coated on insulating base 14 by methods well known in the art such as screen printing and reel to reel fusion. An example of a suitable commercially available material is PD034 formulation an Ag containing resin from Acheson Inc., Michigan. Conductive track 50 are generally formed first on insulating base 14 so they may be at least partially coated with the conductive electrode base 16 for the sensing electrode and the conductive electrode base 21 for the reference electrode in order to make good electrical contact. Typically, conductive track 50 are screen printed on a insulating base 14 such as a polyester backing and cured for about 30 minutes at about 80° C.

The conductive electrode base 16 of sensing electrode 20, the conductive electrode base 21 of reference electrode 30 and the conductive electrode base 17 of passive cover electrode 40 may comprise electrically conductive carbon or graphite, copper, silver, gold, platinum, nickel, stainless steel, iron and other conductive materials and mixtures thereof. The conductive base for each electrode may differ in composition within the same biosensor 10. Formulations of electrically conductive carbon or graphite containing polymeric materials such as the electrically conductive inks available from Acheson Inc., Michigan are preferred as they are readily available and can be uniformly spread on insulating base 14 to form a thin layer and can be easily blended with a redox mediator. Conductive electrode bases 16, 21 and 17 can be applied to insulating base 14 by methods known in the art such as the reel to reel fusion or screen printing.

Sensing electrode 20 may be formed by either blending redox mediators and enzymes with a conductive electrode base composition and then coating this mixture on insulating base 14 or by coating a conductive electrode base composition on insulating base 14 to form conductive electrode base 16 and then coating the surface of conductive electrode base 16 with a formulation containing redox mediators and enzymes. Coating the surface of conductive electrode 16 is preferred as this process is more efficient in manufacturing and produces a more uniform product. Any coating method known in the art such as reel to reel fusion or screen printing or spread coating may be used.

The composition of the mediator-enzyme formulation coating 18 coated on conductive electrode base 16 will very according to the substance that biosensor 10 is intended to determine. For a biosensor intended for the quantitative analysis of glucose in biological fluids such as blood and urine compounds and ions and mixtures of compounds and ions that can function as redox mediators include salts of ferrocyanide (Fe(CN)₆⁻⁴); HCN; I⁻; Co(NH₃)₆⁺⁺; Sn⁺²; S⁻⁻; Tl⁺² and mixtures thereof; methyl viologen, methylene blue, thialene, iodine, dimethylferrocene (DMF) ferricinium, ferrocene monocarboxylic acid (FCOOH), 7,7,8,8-tetracyanoquinodimethane (TCNQ), tetrathiafulvalene (TTF), nickelocence (Nc), N-Methylacridinium (NMA⁺), tetrathiatetracene (TTT), N-methylphenazinium (NMP⁺) hydroquinone, quinhydrone, 3,3′5,5′-tetramethylbenzidine (TMB); 3-methyl-2-benzonthiazolinone hydrazone hydrochloride and 3-dimethylaminobenzoic acid (MBTH-DMAB); o-dianisidine; o-toluidine; sulfonated 2,4-dichloro-phenol plus 4-amino phenazone; benzidine; 3-methyl-2-benzothiozolinone hydrazone plus 3-(dimethylamino) benzoic acid or 2-methoxy-4 allyl phenol; 4-aminoantipyrene-dimethylaniline and 4-aminoantipyrene-4-methoxynaphthol; or mixtures thereof. Ferrocyanide (Fe (CN)₆⁴) salts, particularly K₃fe(CN)₆, are preferred as they generally produce relatively large changes in current flow with changes of glucose concentration over a wide range when used in both the sensing electrode formulation coating 18 and the passive cover electrode formulation coating 24 of biosensor 10. In other embodiments, however, it may be advantageous to use a mediator composition in the sensing electrode formulation coating 18 that differs from that used in the passive cover electrode formulation coating 24 of biosensor 10. Glucose oxidase (GOX) is preferred as the enzyme component of the sensing electrode formulation coating 18 while passive cover electrode formulation coating 24 contains no enzyme in the preferred version of a glucose biosensor embodiment of the present invention. In other embodiments, however, it may be advantageous to use the same or a different enzyme component in passive cover electrode formulation coating 24.

Reference electrode 30 may be formed by coating a conductive electrode base composition on insulation base 14 to form conductive electrode base 21 and then coating the surface of conductive electrode base 21 with a reference electrode formulation 22. Reference electrode formulations such as inks base on Ag/AgCl compositions are generally preferred. Such Formulations comprising electrically conductive graphite, resins and Ag/AgCl are known in the art. A typical formulation is Achson SS 24600, Acheson, Inc, Michigan. Any coating method known in the art such as reel to reel fusion or screen printing may be used.

Reference electrode 30 and sensing electrode 20 are preferably formed on the same backing 14 and arrayed parallel to each to each other as depicted in FIG. 1A. As indicated in FIG. 1A separation 25 separates side-by-side electrode 20 and 30. Separation 25 between electrodes 20 and 30 should preferably be 0.5 mm or less with the proviso that no electrical short circuit is created between electrodes 20 and 30.

Spacers 12 may comprise any relatively thin non-conductive material such as those used for insulating base 14 but electrically insulating adhesive means, such as adhesives and double sided adhesive tape or adhesive laminating tapes are preferred. The preferred material for spacers 12 is a polymeric non-conductive adhesive tape having adhesive on both tape surfaces such as 3M 415 double sided tape. By laminating this type of tape over an array of side-by-side electrode components 51 as depicted in FIG. 1D, reaction zone 33 is easily defined and passive electrode cover 40 can easily be secured in place over the side-by-side components 51. Double stick adhesive tape may be applied directly to conductive electrode bases 16 and 21 of component 51 and sensing electrode formulation coating 18 and reference electrode coating 22 may be applied to the respective exposed surfaces of the conductive electrode base to form sensing electrode 20 and reference electrode 30. Alternatively, conductive electrode bases 16 and 21 may be coated with sensing electrode formulation coating 18 and reference electrode coating 22 respectively and the double stick tape may then be applied on top of the active electrode surfaces to define reaction zone 33. The biosensor of the present invention my have any dimensions and any shapes that are practical for its intended purpose. For example, the width of an individual biosensor, labeled 52 in FIG. 1D, may be any width that is convenient to manufacture and use. A width 52, of about 10 mm is convenient to manufacture and yields a bisensor of dimensions that are easy to use. However, in some applications a smaller or greater width and other dimensions may be preferable. For a width 52 of about 10 mm the separation between the two spacers 12 that form reaction zone 33 is preferably about 4 mm to about 6 mm, most preferably about 5 mm. For a reaction zone 33 having a width 52 of about 10 mm and a length of about 4 mm to about 6 mm, spacers 12 should have a thickness ranging form about 0.01 mm to about 0.2 mm with a thickness of about 0.1 mm preferred to promote a good capillary effect.

Sensing electrode formulation coating 18 comprises an enzyme system, and a redox mediator. The term redox mediator or mediator is herein defined as a substance or substances that facilitate the flow of electrons in a reduction—oxidation reaction, so that the reaction may occur at a lower potential than when such substance or substances are absent. The enzyme system generally comprises an enzyme and a carries with the carries comprising surfactants and stabilizers. The components of the carries assist the action of the enzyme system by promoting a linear correlation of the amperometric response to glucose concentration or by producing greater sensitivity and more reproducible results over a wide range of glucose concentrations. The enzyme system and redox mediator (or redors compound) generally comprises an aqueous mixture having the following composition: GOX about 100 units/ml to about 2000 units/ml, with about 800 units/ml preferred. Sensing electrode formulation 18 further comprises about 0.05% to about 8% (of the weight of the coating), with about 0.5% preferred, of a surfactant such as cholic acid, Triton X-100, polyethylene glycol, sodium lauryl sulfate, sodium lauryl sarcosinate, hydroxypropyl methylcellulose (“Methocel” 40-101 personal care grade), tetrapropylene diplhenyloxide disulphonate sodium salt (“DOWFAX 2A1”), capryloamplhocarboxyropinoate (“MIRALOL J2M-SF”) polyoxyethylene-2-cetyl ether, Surfynol 485, MEGA-8, MEGA-10 and mixtures thereof. A preferred surfactant is MEGA 8. More preferred is a mixture of Surfynol 485 and MEGA-8 which improve shelf stability of the sensor.

Yet another component of sensing electrode formulation 18 is a water soluble or water dispersible aqueous thickening or gelling agent (also known as a stabilizer) which comprises about 0.5% to about 5% of the weight of the coating, with about 1% preferred. Such thickening or gelling agents may comprise various substances such as gelatin, bovine serum albumin, glutamate. L-arginine, Gantrez, mannitol, gum Arabic, polypep (low viscosity), methocel and mixtures thereof used separately or in combination. However, any water soluble or water dispersible aqueous thickening or gelling agent may be used providing it does not interfere with the chemical process which occurs during glucose assay. Gelatin, methocel and mannitol and mixtures thereof are preferred. Most preferred is a 1% concentration of mannitol.

Yet another component comprising electrode formulation coating 18 is a buffering agent to maintain the pH between about 4 and about 8 during glucose analysis. Any buffering agent may be used in any concentration that can maintain the pH in this range providing it does not interfere with the electron transfer reactions of the biosensor. For example, buffers commonly known in the art such as citrate salts succinate salts, tris-(hydroxymethyl) aminomethane, phosphate salts, 2(N-morpholino) ethanesulfonic acid and mixtures thereof may be used. Citrate buffer is preferred to maintain the pH in the preferred range of about 4.5 to about 5.5. The preferred concentration of buffer ranges from about 0.00625 mole to about 0.05 mole per sensor electrode coating 18, with the most preferred concentration ranging from about 0.0125 mole to about 0.025 mole.

Passive cover electrode formulation coating 24 of the preferred embodiment does not contain an enzyme but is otherwise the same as the sensing electrode formulation 18. The following examples further illustrate embodiments the present invention:

### EXAMPLE 1

This example illustrates the performance of the hybrid biosensor 10 of the present invention versus a conventional side-by-side electrode biosensor.

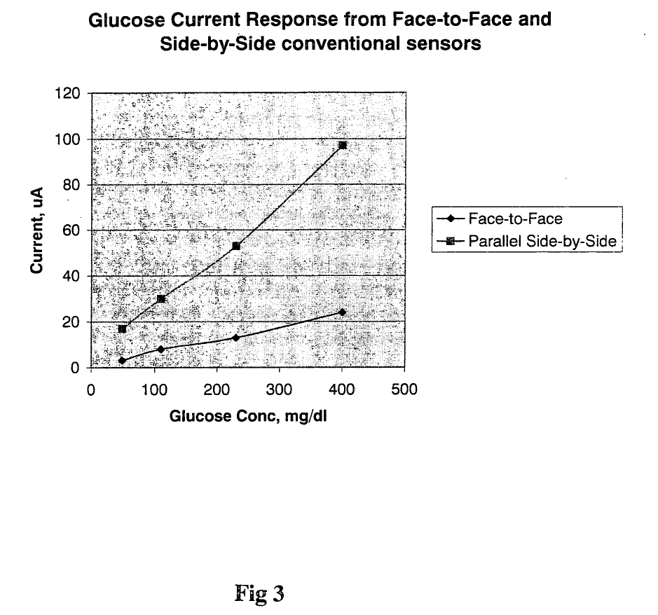

A hybrid biosensor 10 for the detection of glucose was constructed as described about using a polyester film (Mylar®) insulating backing. Using a screen printing process conductive tracks for the reference and sensing electrodes were deposited using an Ag/AgCl conductive ink and conductive electrode bases for the reference and sensing electrodes were deposited using conductive graphite ink. A conductive electrode base for the passive cover electrode was also screen printed on a polyester backing. The conductive electrode base for the reference electrode was coated with 33000 unit/ml Glucose Oxidase, 1M, Potassium ferricyanide, 1% stabilizer, Mannitol, 0.5% Surfactant, Sulfynol to form the reference electrode and the conductive electrode base for the sensing electrode was coated with the same chemicals as the above to form the sensing electrode and the conductive electrode base for the passive cover electrode was coated with 1 M, Potassium Ferricyanide, 0.1%, Bobain Serium Albmin, 1% Stabilizer, Mannitol, 0.1%, Preservative, Methyl Paraben, 1% Surfactant, Sulfynol to form the passive cover electrode. Spacers were formed with double stick tape. An ovoid opening was punched in the cover electrode to permit introduction of sample. A side-by-side sensor having only active electrically wired reference and sensing electrodes was made in the same way except that plain polyester sheet having an ovoid opening was in place of the passive cover electrode. FIG. 3 is a graphical comparison of the performance of an embodiment of the hybrid glucose biosensor of the present invention versus a conventional side-by-side glucose biosensor at an applied potential of 540 mV. As can be clearly seen from FIG. 3 the hybrid biosensor of the present invention has far greater sensitivity since it produces a linear amperometric response at all glucose concentrations that is 5 to 6 times grater than the conventional sensor.

### EXAMPLE 2

This example compares the performance of an embodiment of the hybrid biosensor of the present invention designed to determine glucose concentrations with a glucose biosensor having the face-to-face sandwich configuration disclosed by Guo, et al, in U.S. Pat. No. 6,033,866.

The embodiment of the previously described hybrid biosensor that employs only a glucose oxidase enzyme system was used to determine glucose concentrations at an applied voltage of 540 mV. The face-to-face sandwich configuration using a dual enzyme system comprising glucose oxidase and horse radish peroxidase in a reagent strip was used to determine glucose concentrations at an applied voltage of 125 mV. Two versions of the face-to-face biosensor were used: one used a conductive tape to make electrical connection from the reference electrode to a conductive track in the same plane as the conductive element leading from the sensing electrode so that a conventional electrical connector could be used; a second face-to-face biosensor did not use conductive tape but used an unconventional connector to connect with the conductive elements leading from the sensing and reference electrodes which were in separate planes. The results of this comparison are given in the Table.

| | TABLE | | |
|---|---|---|---|
| | Standard Blood Plasma (“Verichem”) | | |
| | Glucose Performance | | |
| Biosensor Configuration | R² | Intercept μA | Slope (μA/mg/d1) |
| Face-to face sandwich no | 0.9985 | −2.14 | 0.05 |
| conductive tape | | | |
| Face-to face sandwich | 0.998 | 8.0 | 0.42 |
| with conductive tape | | | |
| Hybrid (side-by-side with | 0.999 | 3.0 | 0.42 |
| passive cover electrode) | | | |
| Medisense system | N/A | NA | 0.065 |

R2 represents the linearity of the system.

With reference to the Table it can be seen that use of conductive tape on the face-to face configuration to enable use of a conventional electrical connector causes a 90% decrease in sensitivity as measured by the slope. In contrast to this, the hybrid configuration which naturally enables use of conventional electrical connectors displays the same sensitivity as the face-to-face configuration with conductive tape. The hybrid configuration also produces the same or slightly greater linearity in the relationship between current and glucose concentration as indicated by the R² factor as the face-to-face configuration with conductive tape.

### EXAMPLE 3

This example demonstrates that a hybrid glucose biosensor embodiment of the present invention exhibits linear current response over a wide range of glucose concentrations. Hybrid biosensors 10 designed for the detection of glucose were constructed as described above using a polyester (Mylar®) insulating backing. Spike venous blood glucose solutions containing 30, 50, 120, 240 and 420 mg/dl of glucose were tested at about 540 mV.

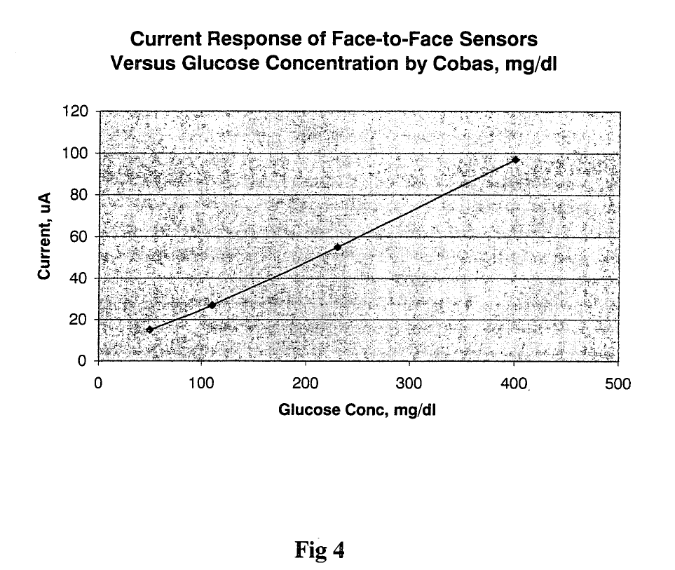

As shown in FIG. 4 a plot of current response versus glucose concentration produces a straight line.

### EXAMPLE 4

This example demonstrates that a hybrid glucose biosensor embodiment of the present invention exhibits linear current response over a wide range of glucose concentrations in EDTA treated whole blood samples.

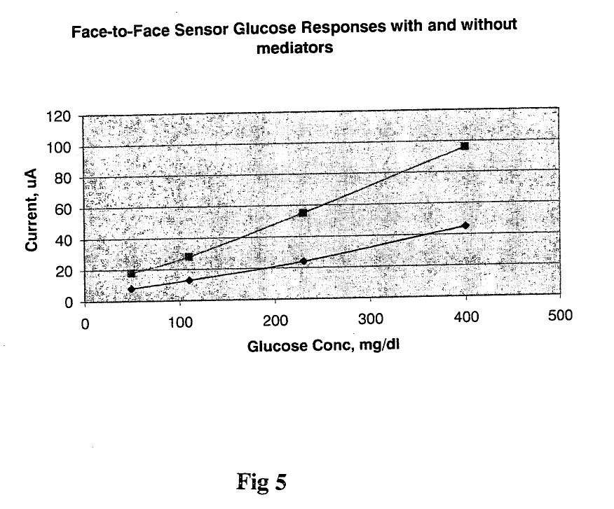

Hybrid biosensors 10 designed for the detection of glucose were constructed as described above using a polyester (Mylar®) insulating backing. EDTA treated whole blood samples containing 67, 101, 208, 354 and 426 mg/dl of glucose were tested at about 540 mV applied voltage. Within about 10 seconds to about 20 seconds. The fast response time of the hybrid biosensor eliminated interference due to lysis of red blood cells which generally occurs after 30 to 45 seconds. As shown in FIG. 5, series 1, a plot of current response versus glucose concentration produces a straight line. The slope of the straight line using unfiltered whole blood samples ranged from about 0.14 to about 0.17 μA/mg/dl depending on the biosensor batch. Although overall sensitivity is reduced when whole blood is used, the hybrid biosensor still produces linear results over a wide range of glucose concentrations comparable with current commercially available glucose sensors (see example 6.)

### EXAMPLE 5

This example demonstrates that the passive cover electrode formulation coating containing a mediator significantly enhances the response of the hybrid biosensor.

Hybrid biosensors 10 designed for the detection of glucose were constructed as described above using a polyester (Mylar®) insulating backing except that the mediator, K₃Fe(CN)₆ was excluded from the passive cover electrode formulation coating 24. These modified hybrid biosensors were then used to determine glucose concentrations in EDTA treated whole blood samples containing 67, 101, 208, 354 and 426 mg/dl of glucose under the same conditions used in Example 4. As shown in FIG. 5 series 2, a plot of current response versus glucose concentration produces a virtually flat straight line. Comparison with the series 1 plot of FIG. 5 clearly shows that sensitivity to glucose concentration is greatly enhanced by the presence of mediator in the passive cover electrode.

### EXAMPLE 6

This example demonstrates that a hybrid glucose biosensor embodiment of the present invention produces substantially the same results in determination of glucose concentration in whole blood as a commercially available biosensor that uses an optical procedure.

Hybrid biosensor 10, designed for the detection of glucose and having mediator in both the sensing electrode and in the passive cover electrode was constructed as described previously using a polyester (Mylar®) insulating backing.

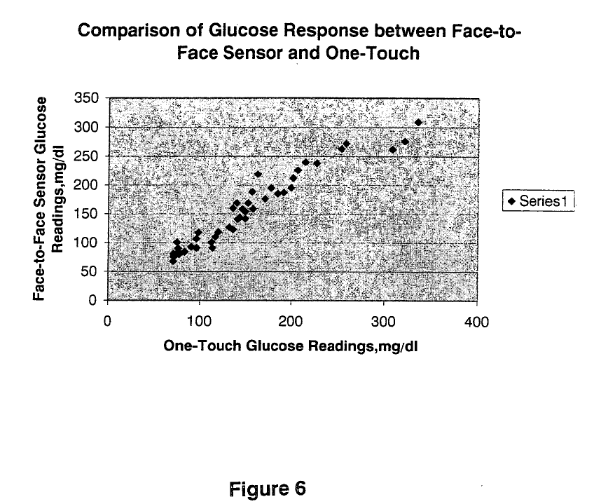

Glucose concentrations in whole blood were determined using a wide range of glucose concentrations in EDTA treated whole blood samples and fingerstick whole blood sample. For each determination of glucose concentration using an amperometric hybrid biosensor of the present invention a determination of glucose concentration on the same sample was made with the Lifescan One Touch Glucose Tester. The glucose concentrations obtained by each method plotted on a concentration vs concentration scatter diagram, FIG. 6, cluster around a well defined straight line with γ=0.989 (n=117) demonstrating a close linear correlation between the results obtained by a glucose biosensor embodiment of the present invention and a commercially accepted blood glucose test.

The invention has been described using exemplary preferred embodiments. However, for those skilled in this field, the preferred embodiments can be easily adapted and modified to suit additional applications without departing from the spirit and scope of this invention. Thus, it is to be understood that the scope of the invention is not limited to the disclosed embodiments. On the contrary, it is intended to cover various modifications and similar arrangements based upon the same operating principle. The scope of the claims, therefore, should be accorded the broadest interpretations so as to encompass all such modifications and similar arrangements.

1. An amperometric sensor for the determination of glucose in aqueous media comprising:
a sensing electrode, the sensing electrode comprising:
a non-conductive support member coated with an electrically conductive layer wherein the electrically conductive layer is further coated with a sensing electrode formulation comprising a first redox mediator, glucose oxidase, at least one surfactant, at least one stabilizer and a buffering agent to maintain a pH from about 4 to about 8;
a reference electrode comprising:
a non-conductive support member coated with an electrically conductive layer wherein the electrically conductive layer is further coated with a reference electrode formation comprising Ag/AgCl dispersed in a resin formation;
a passive cover electrode comprising an electrically conductive layer on a non-conductive support member, with the electrically conductive layer coated with a passive cover electrode formulation comprising a second redox mediator, and with the passive cover electrode having an opening; and
non-electrically conductive spacers disposed parallel at the tops of the sensing electrode formulation and the reference electrode formation so that the sensing electrode and the reference electrode do not come into physical contact with the passive cover electrode when the latter is placed over to define a reaction zone in-between the spacers;
with the electrically conducting layer of the sensing electrode and the electrically conductive layer of the reference electrode arrayed side-by side; and with the passive cover electrode placed over the side-by-side sensing electrode and reference electrode so that the active surface of the passive cover electrode opposes with the active surfaces of the side-by-side electrodes such that the opening exposes a portion of the electrically conducting layer of the sensing electrode and a portion of the electrically conductive layer of the reference electrode that is separated by a separation within the reaction zone to receive the aqueous media.

2. The sensor of claim 1 wherein the non-conductive support member of the sensor electrode further comprises an electrically conductive track extended from and connected to the electrically conductive layer.

3. The sensor of claim 2 wherein the non-conductive support member of the reference electrode further comprises an electrically conductive track extended from and connected to the electrically conductive layer.

4. The sensor of claim 3 wherein the electrically conductive tracks comprise an electrically conductive material selected from the group consisting of metallic silver, silver salts or mixtures thereof, conductive graphite or carbon, copper, platinum, cobalt, nickel, gold and electrically conductive polymers.

5. The sensor of claim 4 wherein the first redox mediator contained in the sensor electrode formulation is selected from the group consisting of ferrocyanide salts (Fe(CN)₆⁴), HCN, I⁻, Co(NH₃)₆⁺⁺, Sn⁺², S⁻⁻, Tl⁺² and mixtures thereof, methyl viologen, methylene blue, thialene, iodine, dimethylferrocene (DMF) ferricinium, ferrocene monocarboxylic acid (FCOOH), 7,7,8,8-tetracyanoquinodimethane (TCNQ), tetrathiafulvalene (TTF), nickelocence (Nc), N-Methylacridinium (NMA⁺), tetrathiafulvalene (TTT), N-methylphenazinium (NMP⁺) hydroquinone, quinhydrone, 3,3′5,5′-tetramethylbenzidine (TMB); 3-methyl-2-benzonthiazolinone hydrazone hydrochloride and 3-dimethylaminobenzoic acid (MBTH-DMAB); o-dianisidine; o-toluidine; sulfonated 2,4-dichloro-phenol plus 4-amino phenazone; benzidine; 3-methyl-2-benzothiozolinone hydrazone plus 3-(dimethylamino) benzoic acid or 2-methoxy-4 allyl phenol; 4-aminoantipyrene-dimethylaniline and 4-aminoantipyrene-4-methoxynaphthol; and mixtures thereof.

6. The sensor of claim 5 wherein the surfactant is selected from the group consisting of cholic acid, Triton X-100, polyethylene glycol, sodium lauryl sulfate, sodium lauryl sarcosinate, hydroxypropyl methylcellulose (“Methocel” 40-101 personal care grade), tetrapropylene diplhenyloxide disulphonate sodium salt (“DOWFAX 2A1”), capryloamplhocarboxyropinoate (“MIRALOL J2M-SF”) polyoxyethylene-2-cetyl ether, Surfynol 485, MEGA-8, MEGA-10 and mixtures thereof.

7. The sensor of claim 6 wherein the stabilizer is selected from the group consisting of gelatin, bovine serum albumin, glutamate. L-arginine, Gantrez, mannitol, gum Arabic, low viscosity polypep, methocel and mixtures thereof used separately or in combination

8. The sensor of claim 7 wherein the buffer is selected from the group consisting of citrate salts succinate salts, tris-(hydroxymethyl) aminomethane, phosphate salts, 2(N-morpholino) ethanesulfonic acid and mixtures thereof.

9. The sensor of claim 8 wherein the electrically conductive layers of the sensing electrode, the reference electrode and the passive cover electrode comprise electrically conductive carbon or graphite, copper, silver, gold, platinum, nickel, stainless steel, iron and mixtures thereof.

10. The sensor of claim 9 wherein the second redox mediator is the same as or different from that of the first redox mediator used in the sensing electrode.

11. The sensor of claim 1 wherein the non-conductive support member comprises a cohesive non-conductor selected from the group consisting of any non-conductive film, sheet forming polymeric material, ceramics, glass, paper, and cardboard.

12. The sensor of claim 11 wherein the sheet forming polymeric material is selected from the group consisting of polyvinyl chloride, polyester, polycarbonate, vinyl acetate copolymer, nylon, poly (1,4-butleneterephthalate), cellulose propionate, ethylene/acrylic acid copolymer, polybutadiene, polyethylene, polypropylene, polyimide, acrylic film, polyurethane, polystyrene, and polyvinyl fluoride.

13. The sensor of claim 1 wherein the spacers comprises any polymeric non-conductive material such as adhesives and double sided adhesive tape or adhesive laminating tapes.

14. An amperometric sensor for the determination of glucose in aqueous media comprising:
a sensing electrode, the sensing electrode comprising:
a non-conductive support member coated with an electrically conductive layer having an electrically conductive track extended therefrom wherein the electrically conductive layer is further coated with a sensing electrode formulation comprising a redox mediator, glucose oxidase, at least one surfactant, at least one stabilizer and a buffering agent to maintain a pH from about 4 to about 8;
a reference electrode comprising:
a non-conductive support member coated with an electrically conductive layer having an electrically conductive track extended therefrom wherein the electrically conductive layer is further coated with a reference electrode formation comprising Ag/AgCl dispersed in a resin formation;
a passive cover electrode comprising an electrically conductive layer on a non-conductive support member, with the electrically conductive layer coated with a passive cover electrode formulation comprising the redox mediator as used in the sensing electrode, and with the passive cover electrode having an opening; and
non-electrically conductive spacers disposed parallel at the tops of the sensing electrode formulation and the reference electrode formation so that the sensing electrode and the reference electrode do not come into physical contact with the passive cover electrode when the latter is placed over to define a reaction zone in-between the spacers;
with the electrically conducting layer of the sensing electrode and the electrically conductive layer of the reference electrode arrayed side-by side; and with the passive cover electrode placed over the side-by-side sensing electrode and reference electrode so that the active surface of the passive cover electrode opposes with the active surfaces of the side-by-side electrodes such that the opening exposes only a portion of the electrically conducting layer of the sensing electrode within the reaction zone to receive the aqueous media.

15. The sensor of claim 14 wherein the non-conductive support member of the sensor electrode further comprises an electrically conductive track extended from and connected to the electrically conductive layer.

16. The sensor of claim 15 wherein the non-conductive support member of the reference electrode further comprises an electrically conductive track extended from and connected to the electrically conductive layer.

17. The sensor of claim 16 wherein the electrically conductive tracks comprise an electrically conductive material selected from the group consisting of metallic silver, silver salts or mixtures thereof, conductive graphite or carbon, copper, platinum, cobalt, nickel, gold and electrically conductive polymers.

18. The sensor of claim 17 wherein the first redox mediator contained in the sensor electrode formulation is selected from the group consisting of ferrocyanide salts (Fe(CN)₆ ⁻⁴), HCN, I⁻, Co(NH₃)₆⁺⁺, Sn⁺², S⁻⁻, Tl⁺² and mixtures thereof, methyl viologen, methylene blue, thialene, iodine, dimethylferrocene (DMF) ferricinium, ferrocene monocarboxylic acid (FCOOH), 7,7,8,8-tetracyanoquinodimethane (TCNQ), tetrathiafulvalene (TTF), nickelocence (Nc), N-Methylacridinium (NMA⁺), tetrathiafulvalene (TTT), N-methylphenazinium (NMP⁺) hydroquinone, quinhydrone, 3,3′5,5′-tetramethylbenzidine (TMB); 3-methyl-2-benzonthiazolinone hydrazone hydrochloride and 3-dimethylaminobenzoic acid (MBTH-DMAB); o-dianisidine; o-toluidine; sulfonated 2,4-dichloro-phenol plus 4-amino phenazone; benzidine; 3-methyl-2-benzothiozolinone hydrazone plus 3-(dimethylamino) benzoic acid or 2-methoxy-4 allyl phenol; 4-aminoantipyrene-dimethylaniline and 4-aminoantipyrene-4-methoxynaphthol; and mixtures thereof.

19. The sensor of claim 18 wherein the surfactant is selected from the group consisting of cholic acid, Triton X-100, polyethylene glycol, sodium lauryl sulfate, sodium lauryl sarcosinate, hydroxypropyl methylcellulose (“Methocel” 40-101 personal care grade), tetrapropylene diplhenyloxide disulphonate sodium salt (“DOWFAX 2A1”), capryloamplhocarboxyropinoate (“MIRALOL J2M-SF”) polyoxyethylene-2-cetyl ether, Surfynol 485, MEGA-8, MEGA-10 and mixtures thereof.

20. The sensor of claim 19 wherein the stabilizer is selected from the group consisting of gelatin, bovine serum albumin, glutamate. L-arginine, Gantrez, mannitol, gum Arabic, low viscosity polypep, methocel and mixtures thereof used separately or in combination

21. The sensor of claim 20 wherein the buffer is selected from the group consisting of citrate salts succinate salts, tris-(hydroxymethyl) aminomethane, phosphate salts, 2(N-morpholino) ethanesulfonic acid and mixtures thereof.

22. The sensor of claim 21 wherein the electrically conductive layers of the sensing electrode, the reference electrode and the passive cover electrode comprise electrically conductive carbon or graphite, copper, silver, gold, platinum, nickel, stainless steel, iron and mixtures thereof.

23. The sensor of claim 22 wherein the second redox mediator is the same as or different from that of the first redox mediator used in the sensing electrode.

24. The sensor of claim 14 wherein the non-conductive support member comprises a cohesive non-conductor selected from the group consisting of any non-conductive film, sheet forming polymeric material, ceramics, glass, paper, and cardboard.

25. The sensor of claim 24 wherein the sheet forming polymeric material is selected from the group consisting of polyvinyl chloride, polyester, polycarbonate, vinyl acetate copolymer, nylon, poly (1,4-butleneterephthalate), cellulose propionate, ethylene/acrylic acid copolymer, polybutadiene, polyethylene, polypropylene, polyimide, acrylic film, polyurethane, polystyrene, and polyvinyl fluoride.

26. The sensor of claim 14 wherein the spacers comprises any polymeric non-conductive material such as adhesives and double sided adhesive tape or adhesive laminating tapes.

27. A method for assaying a sample for determination of the concentration of glucose in an aqueous medium, comprising the steps of:
a) providing an amperometric biosensor for glucose comprising:
a sensing electrode, the sensing electrode comprising:
a non-conductive support member coated with an electrically conductive layer wherein the electrically conductive layer is further coated with a sensing electrode formulation comprising a first redox mediator, glucose oxidase, at least one surfactant, at least one stabilizer and a buffering agent to maintain a pH from about 4 to about 8;
a reference electrode comprising:
a non-conductive support member coated with an electrically conductive layer wherein the electrically conductive layer is further coated with a reference electrode formation comprising Ag/AgCl dispersed in a resin formation;
a passive cover electrode comprising an electrically conductive layer on a non-conductive support member, with the electrically conductive layer coated with a passive cover electrode formulation comprising a second redox mediator, and with the passive cover electrode having an opening; and
non-electrically conductive spacers disposed parallel at the tops of the sensing electrode formulation and the reference electrode formation so that the sensing electrode and the reference electrode do not come into physical contact with the passive cover electrode when the latter is placed over to define a reaction zone in-between the spacers;
with the electrically conducting layer of the sensing electrode and the electrically conductive layer of the reference electrode arrayed side-by side; and with the passive cover electrode placed over the side-by-side sensing electrode and reference electrode so that the active surface of the passive cover electrode opposes with the active surfaces of the side-by-side electrodes such that the opening exposes a portion of the electrically conducting layer of the sensing electrode and a portion of the electrically conductive layer of the reference electrode that is separated by a separation within the reaction zone to receive the aqueous media.
b) introducing a sample into the opening of the passive cover electrode;
c) maintaining a potential of about −450 mV across the sensing electrode and the reference electrode;
d) measuring the current passing between the sensing electrode and the reference electrode; and
e) comparing the current measured to a calibration curve of the concentration of glucose versus current at the potential used in step c) to obtain the concentration of glucose in the sample.

28. The method of claim 27 wherein the non-conductive support member of the sensor electrode of step a) further comprises an electrically conductive track extended from and connected to the electrically conductive layer.

29. The method of claim 28 wherein the non-conductive support member of the reference electrode of step a) further comprises an electrically conductive track extended from and connected to the electrically conductive layer.

30. The method of claim 29 wherein the electrically conductive tracks of step a) comprise an electrically conductive material selected from the group consisting of metallic silver, silver salts or mixtures thereof, conductive graphite or carbon, copper, platinum, cobalt, nickel, gold and electrically conductive polymers.

31. The method of claim 30 wherein the first redox mediator contained in the sensor electrode formulation of step a) is selected from the group consisting of ferrocyanide salts (Fe(CN)₆⁻⁴), HCN, I⁻, Co(NH₃)₆⁺⁺, Sn⁺², S⁻⁻, Tl⁺² and mixtures thereof, methyl viologen, methylene blue, thialene, iodine, dimethylferrocene (DMF) ferricinium, ferrocene monocarboxylic acid (FCOOH), 7,7,8,8-tetracyanoquinodimethane (TCNQ), tetrathiafulvalene (TTF), nickelocence (Nc), N-Methylacridinium (NMA⁺), tetrathiafulvalene (TTT), N-methylphenazinium (NMP⁺) hydroquinone, quinhydrone, 3,3′5,5′-tetramethylbenzidine (TMB); 3-methyl-2-benzonthiazolinone hydrazone hydrochloride and 3-dimethylaminobenzoic acid (MBTH-DMAB); o-dianisidine; o-toluidine; sulfonated 2,4-dichloro-phenol plus 4-amino phenazone; benzidine; 3-methyl-2-benzothiozolinone hydrazone plus 3-(dimethylamino) benzoic acid or 2-methoxy-4 allyl phenol; 4-aminoantipyrene-dimethylaniline and 4-aminoantipyrene-4-methoxynaphthol; and mixtures thereof.

32. The method of claim 31 wherein the surfactant of step a) is selected from the group consisting of cholic acid, Triton X-100, polyethylene glycol, sodium lauryl sulfate, sodium lauryl sarcosinate, hydroxypropyl methylcellulose (“Methocel” 40-101 personal care grade), tetrapropylene diplhenyloxide disulphonate sodium salt (“DOWFAX 2A1”), capryloamplhocarboxyropinoate (“MIRALOL J2M-SF”) polyoxyethylene-2-cetyl ether, Surfynol 485, MEGA-8, MEGA-10 and mixtures thereof.

33. The method of claim 32 wherein the stabilizer of step a) is selected from the group consisting of gelatin, bovine serum albumin, glutamate. L-arginine, Gantrez, mannitol, gum Arabic, low viscosity polypep, methocel and mixtures thereof used separately or in combination

34. The method of claim 33 wherein the buffer of step a) is selected from the group consisting of citrate salts succinate salts, tris-(hydroxymethyl) aminomethane, phosphate salts, 2(N-morpholino) ethanesulfonic acid and mixtures thereof.

35. The method of claim 34 wherein the electrically conductive layers of the sensing electrode, the reference electrode and the passive cover electrode of step a) comprise electrically conductive carbon or graphite, copper, silver, gold, platinum, nickel, stainless steel, iron and mixtures thereof.

36. The method of claim 35 wherein the second redox mediator of step a) is the same as or different from that of the first redox mediator used in the sensing electrode.

37. The method of claim 36 wherein the non-conductive support member of step a) comprises a cohesive non-conductor selected from the group consisting of any non-conductive film, sheet forming polymeric material, ceramics, glass, paper, and cardboard.

38. The method of claim 37 wherein the sheet forming polymeric material is selected from the group consisting of polyvinyl chloride, polyester, polycarbonate, vinyl acetate copolymer, nylon, poly (1,4-butleneterephthalate), cellulose propionate, ethylene/acrylic acid copolymer, polybutadiene, polyethylene, polypropylene, polyimide, acrylic film, polyurethane, polystyrene, and polyvinyl fluoride.

39. The method of claim 38 wherein the spacers of step a) comprises any polymeric non-conductive material such as adhesives and double sided adhesive tape or adhesive laminating tapes.

40. A method for assaying a sample for determination of the concentration of glucose in an aqueous medium, comprising the steps of:
a) providing an amperometric biosensor for glucose comprising:
a sensing electrode, the sensing electrode comprising:
a non-conductive support member coated with an electrically conductive layer wherein the electrically conductive layer is further coated with a sensing electrode formulation comprising a first redox mediator, glucose oxidase, at least one surfactant, at least one stabilizer and a buffering agent to maintain a pH from about 4 to about 8;
a reference electrode comprising:
a non-conductive support member coated with an electrically conductive layer wherein the electrically conductive layer is further coated with a reference electrode formation comprising Ag/AgCl dispersed in a resin formation;
a passive cover electrode comprising an electrically conductive layer on a non-conductive support member, with the electrically conductive layer coated with a passive cover electrode formulation comprising a second redox mediator, and with the passive cover electrode having an opening; and
non-electrically conductive spacers disposed parallel at the tops of the sensing electrode formulation and the reference electrode formation so that the sensing electrode and the reference electrode do not come into physical contact with the passive cover electrode when the latter is placed over to define a reaction zone in-between the spacers;
with the electrically conducting layer of the sensing electrode and the electrically conductive layer of the reference electrode arrayed side-by side; and with the passive cover electrode placed over the side-by-side sensing electrode and reference electrode so that the active surface of the passive cover electrode opposes with the active surfaces of the side-by-side electrodes such that the opening exposes only a portion of the electrically conducting layer of the sensing electrode within the reaction zone to receive the aqueous media;
b) introducing a sample into the opening of the passive cover electrode;
c) maintaining a potential of about −450 mV across the sensing electrode and the reference electrode;
d) measuring the current passing between the sensing electrode and the reference electrode; and
e) comparing the current measured to a calibration curve of the concentration of glucose versus current at the potential used in step c) to obtain the concentration of glucose in the sample.

41. The method of claim 40 wherein the non-conductive support member of the sensor electrode of step a) further comprises an electrically conductive track extended from and connected to the electrically conductive layer.

42. The method of claim 41 wherein the non-conductive support member of the reference electrode of step a) further comprises an electrically conductive track extended from and connected to the electrically conductive layer.

43. The method of claim 42 wherein the electrically conductive tracks of step a) comprise an electrically conductive material selected from the group consisting of metallic silver, silver salts or mixtures thereof, conductive graphite or carbon, copper, platinum, cobalt, nickel, gold and electrically conductive polymers.

44. The method of claim 43 wherein the first redox mediator contained in the sensor electrode formulation of step a) is selected from the group consisting of ferrocyanide salts (Fe(CN)₆⁻⁴), HCN, I⁻, Co(NH₃)₆⁺⁺, Sn⁺², S⁻⁻, Tl⁺² and mixtures thereof, methyl viologen, methylene blue, thialene, iodine, dimethylferrocene (DMF) ferricinium, ferrocene monocarboxylic acid (FCOOH), 7,7,8,8-tetracyanoquinodimethane (TCNQ), tetrathiafulvalene (TTF), nickelocence (Nc), N-Methylacridinium (NMA⁺), tetrathiafulvalene (TTT), N-methylphenazinium (NMP⁺) hydroquinone, quinhydrone, 3,3′5,5′-tetramethylbenzidine (TMB); 3-methyl-2-benzonthiazolinone hydrazone hydrochloride and 3-dimethylaminobenzoic acid (MBTH-DMAB); o-dianisidine; o-toluidine; sulfonated 2,4-dichloro-phenol plus 4-amino phenazone; benzidine; 3-methyl-2-benzothiozolinone hydrazone plus 3-(dimethylamino) benzoic acid or 2-methoxy-4 allyl phenol; 4-aminoantipyrene-dimethylaniline and 4-aminoantipyrene-4-methoxynaphthol; and mixtures thereof.

45. The method of claim 44 wherein the surfactant of step a) is selected from the group consisting of cholic acid, Triton X-100, polyethylene glycol, sodium lauryl sulfate, sodium lauryl sarcosinate, hydroxypropyl methylcellulose (“Methocel” 40-101 personal care grade), tetrapropylene diplhenyloxide disulphonate sodium salt (“DOWFAX 2A1”), capryloamplhocarboxyropinoate (“MIRALOL J2M-SF”) polyoxyethylene-2-cetyl ether, Surfynol 485, MEGA-8, MEGA-10 and mixtures thereof.

46. The method of claim 45 wherein the stabilizer of step a) is selected from the group consisting of gelatin, bovine serum albumin, glutamate. L-arginine, Gantrez, mannitol, gum Arabic, low viscosity polypep, methocel and mixtures thereof used separately or in combination

47. The method of claim 46 wherein the buffer of step a) is selected from the group consisting of citrate salts succinate salts, tris-(hydroxymethyl) aminomethane, phosphate salts, 2(N-morpholino) ethanesulfonic acid and mixtures thereof.

48. The method of claim 47 wherein the electrically conductive layers of the sensing electrode, the reference electrode and the passive cover electrode of step a) comprise electrically conductive carbon or graphite, copper, silver, gold, platinum, nickel, stainless steel, iron and mixtures thereof.

49. The method of claim 48 wherein the second redox mediator of step a) is the same as or different from that of the first redox mediator used in the sensing electrode.

50. The method of claim 49 wherein the non-conductive support member of step a) comprises a cohesive non-conductor selected from the group consisting of any non-conductive film, sheet forming polymeric material, ceramics, glass, paper, and cardboard.

51. The method of claim 50 wherein the sheet forming polymeric material is selected from the group consisting of polyvinyl chloride, polyester, polycarbonate, vinyl acetate copolymer, nylon, poly (1,4-butleneterephthalate), cellulose propionate, ethylene/acrylic acid copolymer, polybutadiene, polyethylene, polypropylene, polyimide, acrylic film, polyurethane, polystyrene, and polyvinyl fluoride.

52. The method of claim 51 wherein the spacers of step a) comprises any polymeric non-conductive material such as adhesives and double sided adhesive tape or adhesive laminating tapes.
